(19) United States
(12) Patent Application Publication
(10) Pub. No.: US 2026/0026980 A1
(43) Pub. Date: Jan. 29, 2026

(54) AN ABSORBENT ARTICLE HAVING STANDING GATHERS AND LEG CUFFS

(71) Applicant: Essity Hygiene and Health Aktiebolag, Göteborg (SE)

(72) Inventors: Helena CORNELIUSSON, Göteborg (SE); Lisa NILSSON, Göteborg (SE); Emelie ADIELS, Göteborg (SE); Yasha ASAYESH, Göteborg (SE)

(73) Assignee: Essity Hygiene and Health Aktiebolag, Göteborg (SE)

(21) Appl. No.: 18/881,481

(22) PCT Filed: Jul. 7, 2022
(86) PCT No.: PCT/EP2022/068932

§ 371 Date: Jan. 6, 2025

(51) Int. Cl. A61F 13/511; A61F 13/15; A61F 13/49; A61F 13/56

(52) U.S. Cl. CPC A61F 13/51104; A61F 13/4902; A61F 13/5638; A61F 2013/15422; A61F 2013/49028

(57) ABSTRACT

An absorbent article including a liquid permeable topsheet, a liquid impermeable backsheet and an absorbent core has a nonwoven outer cover layer which overlies the backsheet, forms an outer surface, and has side portions which extend laterally outside the backsheet and are formed into leg cuff folds. The leg cuff folds are elasticated by spaced-apart elastic elements. A pair of nonwoven barrier cuff strips is arranged with a standing gather portion forming part of a standing gather, the barrier cuff strip being attached to the topsheet along a hinge line and having one or more pre-tensioned elastic elements arranged along a free standing gather edge. A pair of leg cuffs is arranged outward of the standing gathers and include the folded-back, elasticated side portion of the outer cover layer and a leg cuff portion of the corresponding nonwoven barrier strip. The leg cuff portion overlies at least one elastic element.

### TECHNICAL FIELD

The disclosure pertains to an absorbent article having first and second waist edges and first side edges and comprising a liquid permeable topsheet, a liquid impermeable backsheet and an absorbent core disposed between the topsheet and the backsheet. A nonwoven outer cover layer overlays the backsheet and forms an outer surface of the absorbent article. The article further comprises a pair of elasticated standing gathers and a pair of elasticated leg cuffs. A pair of side flaps are arranged at one of the waist edges, the side flaps being provided with fasteners for fastening the absorbent article in a pant-like fashion around the body of a wearer.

### BACKGROUND

Disposable absorbent articles, such as disposable diapers which are applied to the lower body of a wearer and fastened around the wearer's body as a pair of underpants are commonly provided with elastic elements to create fluid barriers and to improve body conformance of the articles. A good body-conforming fit of a disposable absorbent article around the waist and legs of a wearer of the article ensures that the article is held in close contact with the body of the wearer, thereby reducing the risk of fluid leaking out of the absorbent article. Additional leakage protection may be obtained by arranging elasticated standing gathers, also known as “raised barriers” or “inner barriers” inside the absorbent article.

A further important aspect of articles of this kind is that they are comfortable to wear and that they do not give rise to chafing of the sensitive skin in the genital area. A further concern is that tensioned elastic elements may cause discomfort and red-marking by exerting a high pressure on the skin of a wearer, in particular around the legs of the wearer.

### SUMMARY

Disposable absorbent articles having good leakage security and comfort may be achieved by the features of claim 1. Variations of the disclosure are set out in the dependent claims.

Disclosed herein is an absorbent article having a first waist edge and a second waist edge extending in a transverse direction of the absorbent article and a first side edge and a second side edge extending in a longitudinal direction of the absorbent article, the absorbent article comprising:
a liquid permeable topsheet having a topsheet first side edge and a topsheet second side edge;
a liquid impermeable backsheet having a backsheet first side edge and a backsheet second side edge;
an absorbent core disposed between the topsheet and the backsheet;
a nonwoven outer cover layer overlaying the backsheet and forming an outer surface of the absorbent article;
a pair of nonwoven barrier cuff strips extending in the longitudinal direction and being symmetrically arranged with one barrier cuff strip on each side of a longitudinal centre line, each nonwoven barrier cuff strip comprising a standing gather portion being defined between a free standing gather edge extending in the longitudinal direction and a standing gather hinge line extending in the longitudinal direction, the barrier cuff strip being attached to the topsheet along the standing gather hinge line and having one or more elastic elements arranged along the free standing gather edge;
a pair of standing gathers, the standing gathers comprising the standing gather portions of the barrier cuff strips;
a pair of leg cuffs extending in the longitudinal direction and being symmetrically arranged along the first and second side edges, each leg cuff being arranged outward of the standing gather portion of the corresponding barrier cuff strip; and
   wherein
   each leg cuff comprises:
a side portion of the outer cover layer which extends in the transverse direction outward of the corresponding backsheet side edge, the side portion of the outer cover layer being folded back on itself along a fold line extending in the longitudinal direction and dividing the side portion of the outer cover layer in a first part and a second part which first and second parts together form a leg cuff fold having a free edge defined by the fold line and a leg cuff width in the transverse direction between the fold line in the leg cuff fold and the standing gather hinge line;
a plurality of spaced-apart pre-tensioned elastic elements extending in the longitudinal direction inside the leg cuff fold and being attached between the first part and the second part of the leg cuff fold; and
a leg cuff portion of the corresponding nonwoven barrier strip, the leg cuff portion of the nonwoven barrier cuff strip being defined between the standing gather hinge line and an outer leg cuff edge of the nonwoven barrier cuff strip which outer leg cuff edge of the nonwoven barrier cuff strip is parallel to the fold-line in the leg cuff fold, the leg cuff portion of the nonwoven barrier strip overlying at least one elastic element of the plurality of elastic elements.

An article as disclosed herein may be in the form of an open diaper comprising a liquid permeable topsheet, a liquid impermeable backsheet and an absorbent core disposed between the topsheet and the backsheet. Alternatively, the absorbent article may be a pant-type absorbent article having closed side seams.

An open diaper as disclosed herein may comprise a pair of side flaps arranged at one of the first or second waist edges, each side flap extending in the transverse direction from a proximal attached end to a free distal end and comprising a fastener being arranged at the distal end of the side flap.

The attached end of the side flaps of the absorbent articles as disclosed herein may be attached to the absorbent article between the leg cuff portion of the nonwoven barrier cuff strip and side portion of the outer cover layer. The plurality of elastic elements may not extend longitudinally into the location of the attachment of the side flaps.

An open diaper may alternatively be a belted diaper which is closed by connecting the ends of a belt provided at a waist edge of the article with each other. The absorbent article has a nonwoven outer cover layer which overlies the backsheet and forms an outer surface of the absorbent article and which has side portions extending laterally outside the backsheet and being formed into leg cuff folds, each leg cuff fold being elasticated by a plurality of spaced-apart elastic elements. A pair of nonwoven barrier cuff strips are arranged with a standing gather portion of each barrier cuff strip forming part of a standing gather, the barrier cuff strip being attached to the topsheet along a standing gather hinge line and having one or more pre-tensioned elastic elements arranged along a free standing gather edge. A pair of leg cuffs are arranged outward of the standing gathers, each leg cuff comprising the folded-back, elasticated side portion of the outer cover layer and a leg cuff portion of the corresponding nonwoven barrier strip.

The leg cuff portion of the nonwoven barrier strip overlies at least one elastic element of the plurality of elastic elements.

The barrier cuff strips are directly attached to the topsheet without any intervening components between the barrier cuff strips and the topsheet along the standing gather hinge line.

An article as disclosed herein is skin-friendly and comfortable to wear as all body-contacting surfaces of the article are constituted by soft nonwoven materials. The leg cuffs are particularly soft as the pretensioned elastic elements in the leg cuffs are arranged inside the leg cuff fold, providing the leg cuffs with a folded outer edge. The plurality of spaced-apart pre-tensioned elastic elements cause the nonwoven material in the leg cuff folds to contract and form soft, airy and evenly distributed wrinkles on the surface of the leg cuff. The wrinkled elasticated portion of the leg cuff acts as a cushioning distance means between a wearer's body and the elastic elements. Furthermore, on the inner wearer facing side of the article, the leg cuffs comprise the leg cuff portion of the nonwoven barrier cuff strip, which forms an additional gatherable cushioning layer of the leg cuff. Thus, under the influence of the contractive forces from the elastic elements which are distributed over the width of each leg cuff, the leg cuff portion of the nonwoven barrier cuff strip together with the nonwoven material in the leg cuff fold form a soft, bulky, yet highly body conforming leg cuff.

The leg cuffs are highly breathable, as they are mainly constituted by nonwoven materials. The liquid impermeable backsheet may extend into the leg cuffs in an inner part of each leg cuff by at most 50% of the width of the leg cuff, such as at most 40% of the width of the leg cuff or at most 30% of the width of the leg cuff. The liquid impermeable backsheet may extend into the leg cuffs to a greater part of the width of each leg cuff in smaller sizes of an article than in larger sizes of the article.

It may be preferred that all elastic elements or substantially all elastic elements in each leg cuff are arranged in an outer part of the leg cuff which is free from the liquid impermeable backsheet. Such arrangement of the elastic elements is particularly beneficial for obtaining high breathability in the leg cuff and for taking advantage of the full cushioning effect from the two nonwoven layers on the wearer facing side of the leg cuff.

The elastic elements of the plurality of elastic elements in each leg cuff are preferably applied with an outermost elastic element of the plurality of elastic elements being arranged not more than 5 millimetres from the fold-line in the leg cuff fold, preferably not more than 3 millimetres from the fold-line in the leg cuff fold and most preferred not more 5 than 1 millimetre from the fold-line in the leg cuff fold. The outermost elastic element in each leg cuff may be arranged such that there is no distance between the fold-line in the leg cuff fold and the outermost elastic element, i.e. arranged at the fold-line.

The leg cuffs of the absorbent articles as disclosed herein are well-defined, neat and distinct features which are readily identified by visual inspection of the absorbent article. In prior art absorbent articles, leg elastic is commonly arranged between laterally extending side portions of a topsheet and a backsheet or between side portions of a standing gather material and the backsheet. Such arrangement results in a frilly, floppy non-elasticated side edge portion laterally outside of the elastic elements. When putting on an article having such non-elasticated side portions, it needs to be ascertained that the side-portions are folded out to avoid that the article is applied to the wearer with the elastic side portions folded in over the absorbent body, which may lead to poor fit and ultimately to leakage of body fluids out of the article.

An absorbent article as disclosed herein has soft, corrugated, bulky and well-defined leg cuffs which are correctly positioned when applying the article on a wearer without requiring any manipulation of the leg cuffs. The elasticated leg cuffs lie closely against the wearer's skin and form highly conformable and comfortable outer leakage barriers. By having the elastic elements distributed over the width of the leg cuffs, the risk of the leg cuffs exerting an inappropriately high pressure on the wearer's skin is considerably reduced or even eliminated.

In an absorbent article as disclosed herein, the leg cuff portion of the nonwoven barrier cuff strip may be attached to the upper portion of the leg cuff fold with the leg cuff portion of the nonwoven barrier cuff strip overlying all, or substantially all of the plurality of elastic elements, thereby forming a cushioning layer over the full surface of the elasticated portion of the leg cuff.

In an absorbent article as disclosed herein, the leg cuffs may be relatively wide. Each leg cuff may have a major part of the width of the leg cuff being elasticated by the plurality of elastic elements. A width of the leg cuff may be in the range of from 20 millimetres to 60 millimetres, preferably from 30 millimetres to 60 millimetres and the elastic elements of the plurality of elastic elements may be distributed over an area of the leg cuff having a width which is at least 40% of the width of the leg cuff, preferably at least 50% of the width of the leg cuff.

In an absorbent article as disclosed herein, the first part of the side portion of the outer cover layer may be contiguous with a central part of the outer cover layer which central part of the outer cover layer overlies the backsheet and the second part of the side portion of the outer cover layer may be the folded over part of the side portion of the outer cover layer.

When the article is worn, an upper part of the leg cuff fold will be facing the wearer and a lower part of the leg cuff fold will be a garment-facing part of the leg cuff fold.

The first part of the side portion of the nonwoven outer cover layer, i.e., the part which is contiguous with the central part of the outer cover layer may be the lower part of the leg cuff fold, as seen in a direction from the topsheet and the second part of the side portion of the outer cover layer may be the upper part of the leg cuff fold, as seen in a direction from the topsheet. In this folding configuration the second part of the side portion of the outer cover layer is folded back on top of the first part of the side portion of the outer cover layer, i.e., on the topsheet side of the absorbent article. Such configuration may be beneficial in that there is no distinct boundary between the leg cuff and the central part of the outer cover layer. Firm anchoring of an end portion of the side portion of the outer cover layer may be obtained by securing the end portion of the folded-over second part of the side portion of the outer cover layer between the topsheet and the backsheet.

Alternatively, the first part of the outer cover layer may be the upper part of the leg cuff fold, as seen in a direction from the topsheet and the second part of the side portion of the outer cover layer may be the lower part of the leg cuff fold, as seen in a direction from the topsheet. In this folding configuration the second part of the side portion of the outer cover layer is folded back on below of the first part of the side portion of the outer cover layer, i.e., on the backsheet side of the absorbent article.

In an absorbent article as disclosed herein, the plurality of elastic elements may be constituted by at least 3 elastic elements, preferably at least 4 elastic elements. The plurality of elastic elements may be constituted by 3 to 15 elastic elements, preferably 4 to 10 elastic elements and more preferred 5 to 10 elastic elements and most preferred 5 to 7 elastic elements.

The elastic elements may be elastic threads, strings or elastic bands, preferably elastic threads.

The elastic elements in the plurality of elastic elements in each leg cuff may be spaced apart in the transverse direction by from 1 millimetre to 8 millimetres, preferably from 3 millimetre to 8 millimetres. The elastic elements may be applied with equal spacing over the elasticated area of the leg cuff, or may be applied with different spacing over the width of the leg cuff. By way of example, the elastic elements may be applied with a smaller spacing in an inner part of the leg cuff than in an outer part of the leg cuff to achieve a tighter fit along the inner parts of the leg cuffs.

In an absorbent article as disclosed herein, the elasticated part of each leg cuff may have a width of from 20 millimetres to 40 millimetres, preferably 20 millimetres to 35 millimetres as measured in the transverse direction between the outermost elastic element of the plurality of elastic elements and the innermost elastic element of the plurality of elastic elements.

In a leg cuff in an absorbent article as disclosed herein, the elasticated part of each leg cuff may have a width of from 10 millimetres to 60 millimetres as measured in the transverse direction between the outermost elastic element of the plurality of elastic elements and the innermost elastic element of the plurality of elastic elements, a number of elastic elements in the elasticated part of the leg cuff within the range of from 4 to 7 and a spacing between the elastic elements of from 4 to 6 millimetres.

In an absorbent article as disclosed herein, the outer leg cuff edge of the nonwoven barrier cuff strip may be arranged at or inward of the fold line in the leg cuff fold as seen in the transverse direction. By avoiding that the leg cuff portion of the barrier cuff strip extends laterally outward of the fold line in the leg cuff fold, the side edge of the leg cuff, and of the absorbent article will be defined by the soft, resiliently deformable folded edge of the leg cuff fold.

In an absorbent article as disclosed herein, the outer leg cuff edge of the nonwoven barrier cuff strip may be arranged at least 4 mm inward of the fold line in the leg cuff fold as seen in the transverse direction such as to avoid a fringe close to the side edge of the leg cuff and thereby facilitating the experience of a soft and resilient folded edge.

In an absorbent article as disclosed herein, the leg cuff portion of the nonwoven barrier cuff strip may be attached to the leg cuff fold with an adhesive being uniformly distributed over the attachment area between the nonwoven barrier cuff strip and the leg cuff fold or with uniformly distributed thermo-bonds or ultrasonic bonds. Accordingly, the adhesive is uniformly distributed over an attachment area between the leg cuff portion of the nonwoven barrier cuff strip and the upper part of the side portion of the outer cover layer. The adhesive may be applied by any suitable technique, such as slot-coating. The purpose of creating a uniform attachment between the leg cuff portion of the nonwoven barrier cuff strip and the leg cuff fold is to ascertain that the contractive forces from the spaced-apart pre-tensioned elastic elements in the leg cuff fold are transferred to the leg cuff portion of the nonwoven barrier cuff strip in a uniform manner, causing the leg cuff portion of the nonwoven barrier cuff strip to gather and form corrugations in a controlled manner. One example of achieving this is by adhesive slot coating.

The attachment may be arranged such that the outermost longitudinal edge of the attachment area is at least 5 mm, preferably 10 mm inbound of the leg cuff fold. By arranging the outermost longitudinal edge of the attachment area inbound of the leg cuff fold the outermost part of the nonwoven barrier cuff strip can remain attachment free and its soft characteristics is thus preserved.

The nonwoven materials used for the outer cover material and the barrier cuff strip may include one or more spunbond layers and optionally one or more meltblown layers. The nonwoven material in the nonwoven barrier strip may be different from the nonwoven material of the outer cover material from which the leg cuff fold is formed. The nonwoven barrier cuff strip may be formed from a relatively bulky material. The basis weight of the material in the nonwoven barrier strip may be in the range of from 8 g/m² to 25 g/m². The nonwoven material may be made of thermoplastic polymer fibers or filaments, such as polyolefin, polyesters, polyamide and combinations thereof. The nonwoven material may be formed by any of a variety of different processes, such as spunbonding, airlaying, meltblowing or bonded carded web formation processes. The nonwoven layer may be made of co-formed lamina of nonwoven materials such as an SMS (spunbond/meltblown/spunbond) nonwoven material or an SS (spunbond/spunbond) nonwoven material. The thermoplastic polymer materials in the nonwoven layer may be polypropylene or bicomponent fibers of polypropylene and polyethylene, or of a combination of such materials. It may be preferred that the nonwoven barrier strip is constituted by a hydrophobic material. The nonwoven barrier cuff strip may be a single layer nonwoven material.

The pre-tensioned elastic element or elastic elements which is/are arranged along the free standing gather edge of the nonwoven barrier cuff strip may be arranged in a folded-over portion of the nonwoven barrier cuff strip at the free standing gather edge. The end of the folded-over portion of the nonwoven barrier cuff strip may be attached to the standing gather portion of the nonwoven barrier cuff strip such as to enclose the pre-tensioned elastic element or elastic elements. The pre-tensioned elastic element or elastic elements may also be provided with adhesives. The pre-tensioned elastic element or elastic elements of the standing gather portion of the nonwoven barrier cuff strip may comprise any suitable number of pre-tensioned elastic elements. However, the pre-tensioned elastic element or elastic elements are preferably 1, 2 or 3 elastic elements arranged parallel to each other in an edge portion of the standing gather portion of the nonwoven barrier cuff strip. The spacing between the elastic elements on the standing gather portion of the nonwoven barrier cuff strip may be 2 to 5 millimetres. The elastic element or elements may be in the form of one or more elastic thread, string or band.

The corrugations which are formed in the leg cuffs by the elastic elements are visible both on the inner, wearer-facing side and on the outer garment-facing side of the leg cuffs but have a slightly different appearance due to the leg cuff portion of the nonwoven barrier cuff strip adding to the softness and bulkiness on the inner side of the leg cuffs.

In an absorbent article as disclosed herein, the elastic elements of the plurality of elastic elements may be attached between the first and second parts of the leg cuff fold by means of adhesive. The adhesive may be applied to the elastic elements, whereby the first and second parts of the leg cuff fold are indirectly attached to each other by means of the elastic elements.

In an absorbent article as disclosed herein, one or more elastic elements being arranged in an inner part of each leg cuff fold may have a greater tensile resistance than one or more elastic elements being arranged in an outer part of the leg cuff fold. Alternatively, or in addition thereto, one or more elastic elements being arranged in an inner part of the leg cuff fold may be arranged with a smaller spacing than elastic elements being arranged in an outer part of the leg cuff fold.

The one or more elastic elements being arranged in the inner part of each leg cuff fold may have a linear density (dtex) which is higher than the linear density of the one or more elastic elements being arranged in the outer portion of the leg cuff fold.

Also disclosed herein, as an alternative aspect, is an absorbent article having a first waist edge and a second waist edge extending in a transverse direction of the absorbent article and a first side edge and a second side edge extending in a longitudinal direction of the absorbent article, the absorbent article comprising:
a liquid permeable topsheet having a topsheet first side edge and a topsheet second side edge;
a liquid impermeable backsheet having a backsheet first side edge and a backsheet second side edge;
an absorbent core disposed between the topsheet and the backsheet;
a nonwoven outer cover layer overlaying the backsheet and forming an outer surface of the absorbent article;
a pair of nonwoven barrier cuff strips extending in the longitudinal direction and being symmetrically arranged with one barrier cuff strip on each side of a longitudinal centre line, each nonwoven barrier cuff strip comprising a standing gather portion, being defined between a free standing gather edge extending in the longitudinal direction and a standing gather hinge line extending in the longitudinal direction, the barrier cuff strip being attached to the topsheet along the standing gather hinge line and having one or more pre-tensioned elastic elements arranged along the free standing gather edge;
a pair of standing gathers, the standing gathers comprising the standing gather portions of the barrier cuff strips;
a pair of leg cuffs extending in the longitudinal direction and being symmetrically arranged along the first and second side edges, each leg cuff being arranged outward of the standing gather portion of the corresponding barrier cuff strip; and
   wherein
   each leg cuff comprises:
a tube structure which extends in the transverse direction outward of the standing gather hinge line, having an outer tube edge defining the leg cuff fold edge and a leg cuff width in the transverse direction between the outer tube edge and the standing gather hinge line;
a plurality of spaced-apart pre-tensioned elastic elements extending in the longitudinal direction arranged inside the tube structure,
a leg cuff portion of the corresponding nonwoven barrier cuff strip, the leg cuff portion of the nonwoven barrier cuff strip being defined between the standing gather hinge line and an outer leg cuff edge of the nonwoven barrier cuff strip which outer leg cuff edge of the nonwoven barrier cuff strip is parallel to the outer tube edge of the tube structure, the leg cuff portion of the nonwoven barrier cuff strip overlying at least one elastic element of the plurality of elastic elements.

In such an article the tube structure may be attached to the backsheet and/or the outer cover sheet. The tube structure may be arranged between the leg cuff portion of the corresponding nonwoven barrier cuff strip and the backsheet and/or the outer cover sheet.

The same benefits as previously described herein are achieve with this absorbent article. The leg cuffs are particularly soft as the pretensioned elastic elements in the leg cuffs are arranged inside the tube structure, providing the leg cuffs with a leg cuff folded outer edge. The plurality of spaced-apart pre-tensioned elastic elements cause the nonwoven material in the leg cuff folds to contract and form soft, airy and evenly distributed wrinkles on the surface of the leg cuff.

The backsheet may be of the same width as the outer cover sheet, or it may be narrower than the outer cover sheet width. The backsheet may extend partially into the leg cuff, or not at all. The tube structure may thus be arranged to fully or partially overlay the backsheet, or be arranged fully outside of the side edge of the backsheet in transverse direction such that the tube structure is arranged directly on the outer cover sheet.

The tube structure may be arranged at least partly outside of the outer edge of the outer cover sheet in transverse direction. The leg cuff is thereby made up of three sheets and can be soft and pliable.

The tube structure may be formed by a continuous piece of sheet material being folded around the the plurality of elastic elements, and joined by a longitudinally extending joint to form the tube structure.

The tube structure may be attached to the wearer-facing side of the outer cover sheet. Alternatively, the tube structure may be attached to the garment-facing side of the outer cover sheet.

The alternative leg cuff aspect may be combined with one or more of the features as set out in the above, so as to obtain similar advantages.

For example, the outer leg cuff edge of the nonwoven barrier cuff strip may be arranged at or inward of the outer tube edge as seen in the transverse direction.

Also, the leg cuff portion of the nonwoven barrier cuff strip may be attached to the tube structure with an adhesive being uniformly distributed over the attachment area between the nonwoven barrier cuff strip and the tube structure or with uniformly distributed thermo-bonds or ultrasonic bonds as previously disclosed herein.

Further, the sheet material used for the tube structure may be the same as disclosed above for the outer cover material and the barrier cuff strip. As already disclosed, the leg cuffs are highly breathable as they are mainly constituted by nonwoven materials. The dimensions of the leg cuffs may be the same as already herein disclosed.

Moreover, the plurality of spaced-apart pre-tensioned elastic elements arranged in the tube structure may have the same properties and distribution as previously disclosed herein.

The alternative leg cuff may be arranged in an on open diaper and may comprise a pair of side flaps with fastener, or a belt, as disclosed herein.

### BRIEF DESCRIPTION OF THE DRAWINGS

The absorbent articles as disclosed herein will be further explained hereinafter with reference to the appended drawings wherein:

FIG. 1 shows a planar view of a stretched-out absorbent article in the form of a diaper, seen from the side which is intended to be facing a wearer during use of the diaper;

FIG. 2 shows a cross-section through the diaper in FIG. 1, taken along the line II-II in FIG. 1;

FIG. 3 shows a detail of a leg cuff; such as a leg cuff as shown in FIGS. 1 and 2;

FIG. 4 shows a perspective view of the absorbent article in FIG. 1, ready for application on a wearer;

FIG. 5 shows an alternative cross-section through the diaper in FIG. 1, taken along the line II-II in FIG. 1; and

FIG. 6a-d shows schematic cross section views of embodiments of a leg cuff as disclosed herein.

### DETAILED DESCRIPTION

Different aspects of the absorbent article as disclosed herein will be described more fully hereinafter with reference to the enclosed drawings. The embodiments disclosed herein should not be construed as being limiting to the claimed absorbent article.

It is to be understood that the drawings are schematic and that individual components, such as layers of material are not necessarily drawn to scale.

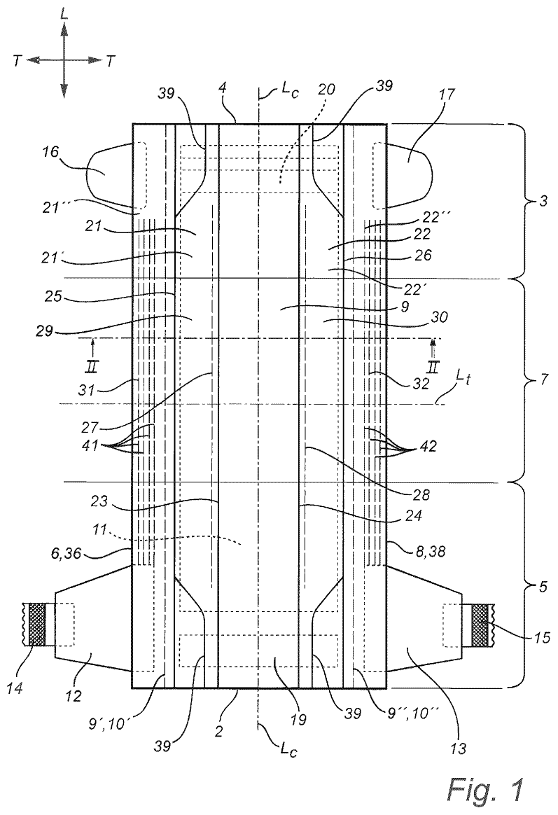

With reference to FIG. 1, there is shown an absorbent article 1 in the form of an open diaper. The absorbent article 1 is shown in FIG. 1 in an unfolded and flat condition with all elastic elements and components in an extended state, such that the absorbent article 1 appears generally two-dimensional. FIG. 1 shows the article from an inner, wearer-facing surface of the article. Any measurements of distances and dimensions are made with the absorbent article in the fully stretched-out, non-gathered condition as shown in FIG. 1.

The pant-type article 1 has a longitudinal direction L and a transverse direction T.

The absorbent article 1 has a first waist edge 2 and a second waist edge 4 and first and second side edges 6, 8. The first waist edge 2 of the absorbent article 1 which is shown in FIG. 1 is a rear waist edge and the second waist edge 4 of the absorbent article 1 is a front waist edge. The first and second waist edges 2, 4 extend in a transverse direction T of the absorbent article 1 and the side edges 6,8 extend in a longitudinal direction L of the absorbent article 1. The absorbent article 1 may be divided in the longitudinal direction L into a from portion 3, a rear portion 5 and a crotch portion 7 located between the front portion 3 and the rear portion 5.

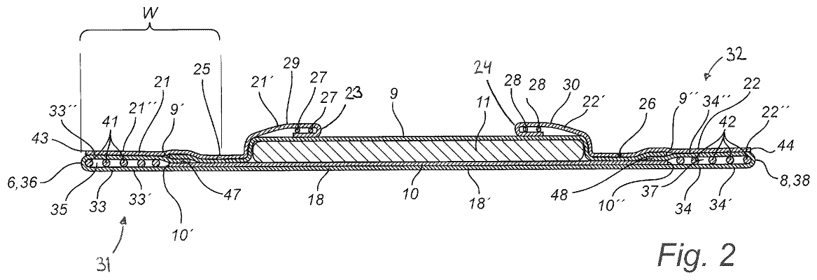

The absorbent article 1 comprises a liquid permeable topsheet 9 and a liquid impermeable backsheet 10. An absorbent core 11 is disposed between the topsheet 9 and the backsheet 10. The topsheet 9 has a topsheet first side edge 9′ and a topsheet second side edge 9″ and the backsheet 10 has a backsheet first side edge 10′ and a backsheet second side edge 10″, as indicated in FIGS. 1 and 2.

The absorbent article 1 further comprises a nonwoven outer cover layer 18 overlaying the backsheet 10 and forming an outer surface of the absorbent article 1.

A pair of nonwoven barrier cuff strips 21, 22 are arranged extending in the longitudinal direction L and are symmetrically arranged with one barrier cuff strip 21, 22 on each side of a longitudinal centre line Lc, perpendicular to a transverse centre line Lt, each nonwoven barrier cuff strip 21, 22 comprising a standing gather portion 21′, 22′. The standing gather portion 21′, 22′ of the nonwoven barrier cuff strip 21, 22 is defined between a free standing gather edge 23, 24 extending in the longitudinal direction L and a standing gather hinge line 25, 26 extending in the longitudinal direction L. A pair of raised elasticated standing gathers 29, 30 comprising the standing gather portions 21′, 22′ of the barrier cuff strips 21, 22 are arranged on the topsheet 9.

Hence, the standing gather portions 21′, 22′ of the barrier cuff strips 21, 22 form parts of the standing gathers 29, 30.

Each barrier cuff strip 21, 22 is directly attached to the topsheet 9 along the standing gather hinge line 25, 26 and has one or more pre-tensioned elastic elements 27, 28 arranged along the free standing gather edge 23, 24. The elastic element or elements may be elastic strings, threads or bands which are applied spaced apart along the free standing gather edge 23, 24. In an absorbent article as disclosed herein the number of elastic elements 27, 28 which are disposed along the standing gather edge, 23, 24 is typically 1, 2 or 3. The elastic elements 27, 28 are typically disposed parallel to each other with a spacing of from 2 to 5 millimetres, with an outermost elastic element arranged at the free standing gather edge 23, 24.

The absorbent article 1 further comprises a pair of leg cuffs 31, 32 extending in the longitudinal direction L and being symmetrically arranged along the first and second side edges 6, 8, each leg cuff 31, 32 being arranged outward of the standing gather portion 21′, 22′ of the corresponding barrier cuff strip 21, 22.

As shown in FIG. 2, the leg cuffs 31, 32 each comprises a side portion 33, 34 of the outer cover layer which extends in the transverse direction outward of the corresponding backsheet side edge 10′, 10″, the side portion 33, 34 of the outer cover layer 18 being folded back on itself along a fold line 36, 38 extending in the longitudinal direction L and dividing the side portion 33, 34 of the outer cover layer 18 in a first part 33′, 34′ and a second part 33″, 34″ which first and second parts 33′,33″; 34′, 34″ together form a leg cuff fold 35, 37 having a free edge defined by the fold line 36, 38 and a leg cuff width W being defined in the transverse direction T between the free edge of the leg cuff fold line 36, 38 and the standing gather hinge line 25, 26.

The first parts 33′, 34′ of the side portions 33, 34 of the outer cover layer 18 are shown in FIG. 2 to be contiguous with a central part 18′ of the outer cover layer 18, which central part 18′ of the outer cover layer 18 overlies the backsheet 10. The second parts 33″, 34″ of the side portions 33, 34 of the outer cover layer 18 are the folded over parts of the side portions 33, 34 of the outer cover layer 18.

In the embodiment shown in the Figures, the first parts 33′, 34′ of the side portions 33, 34 of the outer cover layer 18 constitute lower parts of the leg cuff folds 35, 37, as seen in a direction from the topsheet 9 and the second parts 33″, 34″ of the side portions 33, 34 of the outer cover layer 18 constitute upper parts of the leg cuff folds 35, 37, as seen in a direction from the topsheet 9. The upper parts of the leg cuff folds 35, 37 are folded back on top of the lower parts of the leg cuff folds 35, 37 and are secured between the topsheet 9 and the backsheet 10. Hence, the topsheet 9 and the backsheet 10 are directly connected to each other along side edges of the absorbent core 11 and directly connected to the folded-in parts of the outer cover layer which are secured between the topsheet 9 and the backsheet 10, as seen in FIG. 2. The folded-in parts of the outer cover layer comprise the inner ends 47, 48. The garment-facing side of the backsheet is directly attached to the central part of the outer cover sheet.

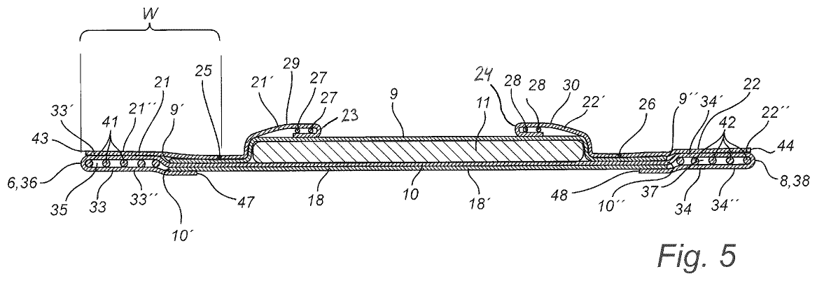

As set out herein, the side portions 33, 34 of the outer cover layer 18 may alternatively be folded such that the first parts 33′, 34′ of the side portions 33, 34 of the outer cover layer 18, which are contiguous with the central part 18′ of the outer cover layer 18 constitute upper parts of the leg cuff folds 35, 37 as seen in a direction from the topsheet and the folded-back second parts 33″, 34″ constitute lower parts of the leg cuff folds 35, 37, as seen in a direction from the topsheet 9. In this configuration, as shown in FIG. 5, the side portions 33, 34 of the outer cover layers 18 are folded back on themselves on the outer garment-facing side of the absorbent article 1.

A plurality of spaced-apart pre-tensioned elastic elements 41, 42 are arranged in each leg cuff fold 35, 37. The elastic elements 41, 42 extend in the longitudinal direction L inside the leg cuff fold 35, 37 and are attached, e.g., by means of adhesive, between the first part 33′, 34′ and the second part 33″, 34″ of the leg cuff fold 35, 37.

The width of the leg cuffs 31, 32 may be in the range of from 20 millimetres to 60 millimetres and the elastic elements of the plurality of elastic elements 41, 42 may be distributed over an area of each leg cuff having a width which is at least 40% of the width of the leg cuff, preferably at least 50% of the width of the leg cuff. The width of the elasticated portion of the leg cuffs 31, 32 may be in the range of from 20 millimetres to 40 millimetres.

The elastic elements of the plurality of elastic elements 41, 42 in each leg cuff 31, 32 may suitably be attached between the first and second parts 33′, 34′; 33″, 34″of the leg cuff fold 35, 37 by means of adhesive. Adhesive may be applied to the elastic elements, and/or to one or both of the first and second parts 33, 34 of the leg cuff fold 35, 37. Alternatively, the elastic elements may be anchored in the leg cuff fold 35, 37 by means of heat welding or ultrasonic welding, e.g., as disclosed in WO2021/037352 A1.

The elastic elements may be elastic threads, bands, strings, etc. All elastic elements may be of the same type and having the same elastic properties. Alternatively, one or more elastic elements being arranged in an outer part of the leg cuff fold 35, 37 may have a greater tensile resistance than one or more elastic elements being arranged in an inner part of the leg cuff fold 35, 37.

The one or more elastic elements in the outer part of the leg cuff fold 35, 37 may have a linear density (dtex) which is higher than the linear density of the one or more elastic elements being arranged in the inner portion of the leg cuff fold 35, 37.

Each leg cuff 31, 32 further comprises a leg cuff portion 21″, 22″ of the corresponding nonwoven barrier strip 21, 22. The leg cuff portion 21″, 22″ of the nonwoven barrier cuff strip 21, 22 is defined between the standing gather hinge line 25, 26 and an outer leg cuff edge 43, 44 of the nonwoven barrier cuff strip 21, 22. The outer leg cuff edge 43, 44 of the nonwoven barrier cuff strip 21, 22 is parallel to the fold-line 36, 38 in the leg cuff fold 35, 37 and overlies at least one elastic element of the plurality of elastic elements 41, 42. In the embodiment shown in the figures, the leg cuff portions 21″, 22″ of the nonwoven barrier strips 21, 22 overlie all elastic elements of the plurality of elastic elements 41, 42 in the leg cuff folds 35, 37 and the outer leg cuff edges 43, 44 of the leg cuff portions 21″, 22″ coincide with the fold lines 36, 38 in the side portions 33, 34 of the outer cover layer 18. It is to be understood that the outer leg cuff edges 43, 44 of the leg cuff portions 21″, 22″ may not coincide with the fold lines 36, 38 and may be arranged inward of the fold lines 36, 38 in the side portions 33, 34 of the outer cover layer 18.

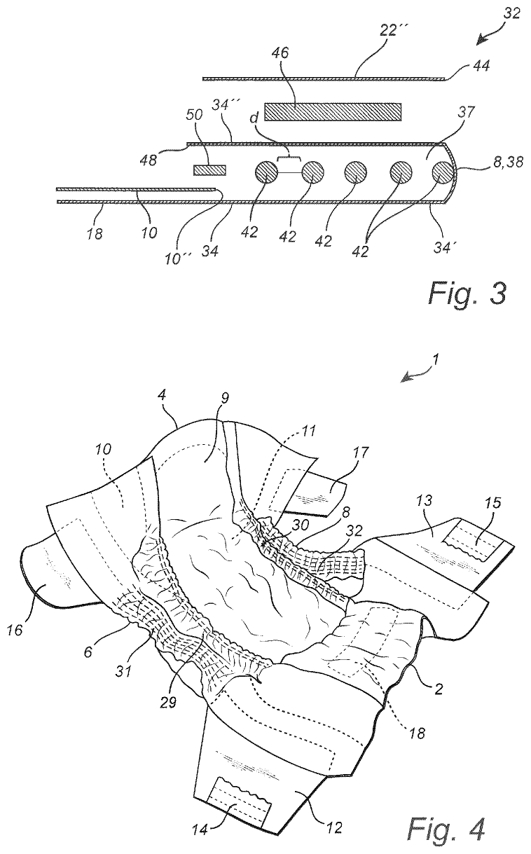

FIG. 3 shows a simplified detail of a portion of the leg cuff 32 which is arranged along the second side edge. A plurality of elastic elements 42, represented by five individual elastic elements are arranged in the leg cuff fold 37 with a spacing d between the individual elastic elements. The elastic elements are shown with equal spacing between all elastic elements. However, the spacing may be different in different parts of the leg cuff 32. By way of example, the spacing between the individual elastic elements may be smaller in an outer part of the leg cuff 32 which is close to the fold line 38 than in an inner part of the leg cuff 32 which is closer to the standing gather hinge line 26 (not shown in FIG. 3).

The leg cuff portion 22″ of the nonwoven barrier cuff strip 22 overlies all elastic elements of the plurality of elastic elements 42 in the leg cuff fold 37 but is only attached to the second, upper, part 34″ of the leg cuff fold 37 in an area of the second part 34″ of the leg cuff fold 37 corresponding to the location of the 4 elastic elements of the plurality of elastic elements 42 which are arranged at a distance from the fold line 38. The leg cuff portion 22″ of the nonwoven barrier cuff strip 22 is attached to the second part 34″ of the leg cuff fold 37 by means of adhesive 46, leaving the outermost part of the leg cuff portion 22″ of the nonwoven barrier cuff strip 22 free from attachments to the leg cuff fold 37. In this manner, no adhesive is arranged immediately adjacent to the side edge of the absorbent article 1. The adhesive attachment between the leg cuff fold and the leg cuff portion 22″ of the nonwoven barrier cuff strip 22 may increase the structural stability of the leg cuff 32 by increasing the bending resistance of the leg cuff. This is an effect of the adhesive attachment which may be beneficial to the overall functionality of the leg cuff, but which may be detrimental to softness of the portion of the leg cuff along the side edge 8. However, it is to be understood that the adhesive area 46 may extend all the way to the fold line 38 in the leg cuff fold 37.

As shown in FIG. 3, which is a highly schematic, exploded view of the leg cuff 32, the outermost elastic element of the plurality of elastic elements 42 is positioned at the fold line 38 such that no excess material is present outward of the outermost elastic element. This may be an ideal positioning of the outermost elastic element. However, due to production tolerances, the positioning of the outermost elastic element in an actual absorbent article 1 may deviate somewhat from the position shown in FIG. 3.

As disclosed herein, the leg cuff portion 22″ of the nonwoven barrier cuff strip 22 may be attached to the leg cuff fold 37 with an adhesive which has been uniformly distributed over the attachment area between the barrier cuff strip 22 and the upper part 34″ of the leg cuff fold 37, creating a uniform attachment between the leg cuff portion 22″ of the nonwoven barrier cuff strip 22 and the leg cuff fold 37 so that the contractive forces from the spaced-apart pre-tensioned elastic elements 42 in the leg cuff fold 37 are transferred to the leg cuff portion 22″ of the nonwoven barrier cuff strip 22 in a substantially equal way over the whole attachment area. Thereby, when the elastic elements contract, the leg cuff portion 22″ of the nonwoven barrier cuff strip 22 gathers together with the material in the leg cuff fold, 37 and forms soft corrugations in the leg cuff 32 in a controlled and neat manner.

As also seen in FIG. 3, the second part 34″ of the leg cuff fold 37 is secured to the backsheet 10 along the backsheet side edge 10″ at an inner end 48 of the second part 34″ of the leg cuff fold 37. As seen in FIG. 2, the inner ends 47, 48 of the second parts 33″, 34″ of the side portions 33, 34 are inserted between the topsheet 9 and the backsheet 10 and are secured between the topsheet 9 and the backsheet 10. The attachment is made by means of adhesive 50. However, it is to be understood that the leg cuff portion 22″ of the nonwoven barrier strip 22 may alternatively be attached by means of thermo-welding or ultrasonic welding. In the embodiment shown in FIG. 3, all elastic elements of the plurality of elastic elements 42 are arranged in a portion of the leg cuff fold 37 where the backsheet 10 is absent. Although, this may be a desired arrangement providing maximum breathability to the leg cuff 32, the backsheet 10 may alternatively extend into the leg cuff 32 such that it overlaps with an innermost elastic element of the plurality of elastic elements 42.

A first side flap 12 is arranged on the rear portion 5 of the absorbent article 1, at the rear waist edge 2, and extends from the first side edge 6 in the transverse direction T of the absorbent article 1 and a second side flap 13 is arranged on the rear portion 5, at the rear waist edge 2, and extends from the second side edge 8 in the transverse direction T of the absorbent article 1. The first and second side flaps 12, 13 are attachment flaps and are provided with fasteners 14, 15 for securing the absorbent article 1 in a pant-like configuration about the lower trunk of a wearer's body. Such attachment flaps are optional features of an absorbent article as disclosed herein e.g., in absorbent articles being pant diapers or belted diapers.

The first and second side flaps 12, 13 are tensioned when putting on the absorbent article and need to be firmly anchored along the side edges 6,8 to ascertain that they do not come off the article when exposed to laterally applied pulling forces. The first and second side flaps 12, 13 may be made from any suitable material as known in the art, such as fibrous nonwoven material. The first and second side flaps 12, 13 may be elastic or may be elastically stretchable, at least in the lateral direction of the absorbent article 1. The first and second side flaps 12, 13 may be breathable and are preferably soft and pliable for providing good body conformance and comfort.

The first and second side flaps 12, 13 which are shown in FIGS. 1 and 4 are provided with fastener elements 14, 15 which may be of any suitable kind such as adhesive or mechanical fasteners. A common type of mechanical fastener is hook-and-loop fasteners.

In the absorbent article 1 in FIGS. 1 and 4, the fastener elements 14, 15 are shown as hook-type fasteners, i.e., fasteners having multiple protruding elements which may engage with a corresponding loop-type fastener such as a loop-type nonwoven material. A loop-type fastener may be provided by a portion of the nonwoven outer cover layer 18 which overlies the backsheet 10 on the outer, garment-facing, surface of the absorbent article 1. Alternatively, a dedicated fastener receiving area 20 formed by a fastener receiving material maybe be arranged on the outer, garment-facing surface of the front portion 3 of the absorbent article 1, as indicated in FIG. 1.

The fastener elements 14, 15 may alternatively be adhesive fastener tapes which are arranged to be attached on a receiving area arranged on the front portion 3 of the absorbent article 1. A further option is that the first and second side flaps 12, 13 are arranged to be attached to third and fourth side flaps arranged at the opposite waist edge. Furthermore, the first and second side flaps 12, 13 may have a lateral extension which is large enough such that the first and second side flaps 12, 13 can be fastened to each other in the manner of a belt. It is further to be understood that in a hook-and-loop type fastener arrangement, the hook-type fastener elements may be arranged on a receiving surface while the loop type fastener elements are arranged on a first and/or a second side flap.

The third and fourth side flaps 16, 17 as shown in the figures are optional to the absorbent article as disclosed herein. The third and fourth side flaps 16,17, may be made from generally the same materials as the first and second side flaps 12, 13. However, elastic materials are less suitable for use in the third and fourth side flaps 16, 17. In the absorbent article 1 in FIGS. 1 and 4, the third and fourth side flaps 16, 17 are positioning flaps serving as grip members when applying the front portion 3 of the absorbent article 1 over a wearer's belly.

As is best seen in FIG. 1, the plurality of elastic elements 41, 42 in the leg cuffs 31, 32 preferably extend only along the parts of the side edges 6, 8 which are located between the first and third side flaps 12, 16 for the first side edge 6 and between the second and fourth side flaps 13, 15 for the second side edge 8, such that the major parts of the leg cuffs 31, 32 are located in the crotch portion 7 of the absorbent article 1. Likewise, the elastic elements 27, 28 in the standing gathers 29, 30 are preferably arranged as shown in FIG. 1 such that the elastic elements 27, 28 do not extend all the way to the end edges 2, 4 of the absorbent article 1. The non-elasticated parts of the standing gathers 29,30 are secured to the topsheet along lines of securement 39. Thereby, under the influence of the elastic forces in the elastic elements 27, 28, the portion of the absorbent article 1 which is located between the standing gathers 29, 30 assumes a pouch-shaped in-use configuration in the crotch portion 7, as shown in FIG. 4.

The absorbent article 1 in FIGS. 1 and 4 further includes an optional waist elastic member 18. The waist elastic member 19 is located at the rear waist edge 2 and extends in the lateral direction along only a part of the rear waist edge 2. Any type of waist elastic may be used, as known in the art. Furthermore, the absorbent article 1 may include waist elastic also at the front portion 3 of the absorbent article 1.

As seen in FIG. 4, when the absorbent article 1 has been taken out of a package and has been folded out ready for application to a wearer, the standing gathers 29, 30 are raised up from the topsheet 9 as a result of the contractive forces exerted on the free standing gather edges 23, 24 by the pre-tensioned elastic elements 27, 28 which are arranged on the standing gathers 29, 30. The elastic elements of the plurality of elastic elements 41, 42 in the leg cuffs 31, 32 gather the nonwoven material in the leg cuff folds 35, 37 as well as the nonwoven material in the leg cuff portions 21″, 22″ of the nonwoven barrier cuff strips 21, 22, thereby creating fluffy and breathable leg cuffs 31, 32 which are resiliently compressible in a thickness direction of the leg cuffs 31, 32 and which have a broad skin-contacting inner surface over which the contractive forces from the plurality of elastic elements 41, 42 in the leg cuffs 31, 32 are distributed. Thereby, the leg cuffs fit closely around the legs of a wearer in a comfortable manner, with virtually no risk of chafing or other discomforts being caused by the leg cuffs.

As further seen in FIG. 4, the broad, elasticated leg cuffs 31, 32 are bulky and shape stable and automatically assumes a correct position when the absorbent article is applied to a wearer.

In the configuration shown in FIG. 5, the leg cuffs 31, 32 each comprises a side portion 33, 34 of the outer cover layer which extends in the transverse direction outward of the corresponding backsheet side edge 10′, 10″, the side portion 33, 34 of the outer cover layer 18 being folded back on itself along a fold line 36, 38 extending in the longitudinal direction L and dividing the side portion 33, 34 of the outer cover layer 18 in a first part 33′, 34′ and a second part 33″, 34″ which first and second parts 33′,33″; 34′, 34″ together form a leg cuff fold 35, 37 having a free edge defined by the fold line 36, 38 and a leg cuff width W being defined in the transverse direction T between the free edge of the leg cuff fold line 36, 38 and the standing gather hinge line 25, 26.

The first parts 33′, 34′ of the side portions 33, 34 of the outer cover layer 18 are shown in FIG. 5 to be contiguous with a central part 18′ of the outer cover layer 18, which central part 18′ of the outer cover layer 18 overlies the backsheet 10.

As shown in FIG. 5, the first parts 33′, 34′ of the side portions 33, 34 of the outer cover layer 18 constitute upper parts of the leg cuff folds 35, 37, as seen in a direction from the topsheet 9 and the second parts 33″, 34″ of the side portions 33, 34 of the outer cover layer 18 constitute lower parts of the leg cuff folds 35, 37, as seen in a direction from the topsheet 9. The lower parts of the leg cuff folds 35, 37 are folded back underneath of the upper part of the leg cuff folds 35, 37 and is attached to the outer cover layer 18.

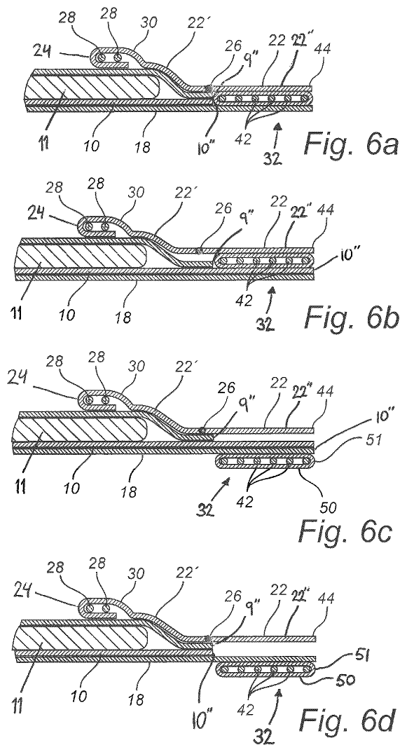

Alternative leg cuffs, comprising a tube structure 50, are shown in the highly schematic FIGS. 6a-d. Elastic elements 42 are arranged inside the tube structure 50. The leg cuff portion 22″ of the nonwoven barrier cuff strip 22 overlay the elastic element of the plurality of elastic elements 42. In FIG. 6a the backsheet 10 is narrower in transversal direction than the outer cover sheet 18, and the tube structure 50 is attached to the outer cover sheet 18. Alternatively, shown in FIG. 6b, the tube structure 50 may be attached to the backsheet. As seen in FIG. 6c-d the tube structure may also be attached to the garment-facing side of the outer cover sheet 18. The tube structure may also be arranged partly outside of the outer edge of outer cover sheet 18 (not shown).

The absorbent core 11 is disposed between the topsheet 9 and the backsheet 10 and is arranged to absorb liquid, such as urine or other body fluids, which has passed through the topsheet 9. The absorbent core 11 may be a single-layer structure or may be a layered structure. The absorbent core 11 may comprise any conventional material suitable for absorbing discharged bodily wastes, such as cellulosic fluff pulp, tissue layers, highly absorbent polymers (super absorbents), absorbent foam materials including hydrogel-foam material, nonwoven materials, or the like. The absorbent core may comprise non-absorbent components such as stiffening elements, shaping elements, binders, etc. Various types of liquid-receiving and liquid distribution materials can also be included in the core.

The absorbent core 11 may be a three-dimensionally shaped core with different amounts of absorbent material in different parts of the core. The core may be provided with features such as embossings, fluid acquisition cavities and fluid distribution channels. Properties of the absorbent core such as density, amount of absorbent material and absorbent capacity may also be varied in different portions of the absorbent core. The absorbent core 11 may comprise a core cover, sandwiching absorbent material between an upper core cover layer and a lower core cover layer. Such core cover may be a tissue material or a nonwoven material and is provided in addition to the topsheet 9 and the backsheet 10.

The liquid permeable topsheet 9 may comprise or consist of a nonwoven material. Other suitable topsheet materials include tow fibres, porous foams, apertured plastic films and laminates and combinations of such materials. The materials which are best suited as topsheet materials are soft and non-irritating to the skin, are readily penetrated by body fluids, and display low rewet of absorbed body fluid.

The liquid impermeable backsheet 10 may be any conventional liquid impermeable material such as a thin plastic film, e. g. a polyethylene or polypropylene film. The backsheet material may be breathable to allow vapour to escape from the absorbent body, while still preventing liquids from passing through the backsheet material.

1. An absorbent article having a first waist edge and a second waist edge extending in a transverse direction of the absorbent article and a first side edge and a second side edge extending in a longitudinal direction of the absorbent article, the absorbent article comprising:
a liquid permeable topsheet having a topsheet first side edge and a topsheet second side edge;
a liquid impermeable backsheet having a backsheet first side edge and a backsheet second side edge;
an absorbent core disposed between the topsheet and the backsheet;
a nonwoven outer cover layer overlaying the backsheet and forming an outer surface of the absorbent article;
a pair of nonwoven barrier cuff strips extending in the longitudinal direction and being symmetrically arranged with one barrier cuff strip on each side of a longitudinal centre line, each nonwoven barrier cuff strip comprising a standing gather portion, being defined between a free standing gather edge extending in the longitudinal direction and a standing gather hinge line extending in the longitudinal direction, the barrier cuff strip being attached to the topsheet along the standing gather hinge line and having one or more pre-tensioned elastic elements arranged along the free standing gather edge;
a pair of standing gathers, the standing gathers comprising the standing gather portions of the barrier cuff strips;
a pair of leg cuffs extending in the longitudinal direction and being symmetrically arranged along the first and second side edges, each leg cuff being arranged outward of the standing gather portion of the corresponding barrier cuff strip; and
wherein
each leg cuff comprises:
a side portion of the outer cover layer which extends in the transverse direction outward of the corresponding backsheet side edge, the side portion of the outer cover layer being folded back on itself along a fold line extending in the longitudinal direction and dividing the side portion of the outer cover layer in a first part and a second part which first and second parts together form a leg cuff fold having a free edge defined by the fold line and a leg cuff width in the transverse direction between the fold line in the leg cuff fold and the standing gather hinge line;
a plurality of spaced-apart pre-tensioned elastic elements extending in the longitudinal direction inside the leg cuff fold and being attached between the first part and the second part of the leg cuff fold; and
a leg cuff portion of the corresponding nonwoven barrier cuff strip, the leg cuff portion of the nonwoven barrier cuff strip being defined between the standing gather hinge line and an outer leg cuff edge of the nonwoven barrier cuff strip which outer leg cuff edge of the nonwoven barrier cuff strip is parallel to the fold line in the leg cuff fold, the leg cuff portion of the nonwoven barrier cuff strip overlying at least one elastic element of the plurality of elastic elements.

2. The absorbent article according to claim 1, wherein the absorbent article further comprises a pair of side flaps arranged at one of the first or second waist edges, each side flap extending in the transverse direction from a proximal attached end to a free distal end and comprising a fastener being arranged at the distal end of the side flap.

3. The absorbent article according to claim 1, wherein the leg cuff portion of each nonwoven barrier cuff strip is attached to the corresponding leg cuff fold with the leg cuff portion of the nonwoven barrier cuff strip overlaying all, or substantially all of the plurality of elastic elements in the leg cuff fold.

4. The absorbent article according to claim 1, wherein the width of each leg cuff is in the range of from 20 millimetres to 60 millimetres and wherein the elastic elements of the plurality of elastic elements in each leg cuff are distributed over an area of the leg cuff having a width in the transverse direction which is at least 40% of the width of the leg cuff.

5. The absorbent article according to claim 1, wherein the first part of the side portion of the outer cover layer is contiguous with a central part of the outer cover layer, which central part of the outer cover layer overlies the backsheet and wherein the second part of the side portion of the outer cover layer is the folded-over part of the side portion of the outer cover layer.

6. The absorbent article according to claim 5, wherein the first part of the side portion of the outer cover layer is a lower part of the leg cuff fold, as seen in a direction from the topsheet and the second part of the side portion of the outer cover layer is an upper part of the leg cuff fold, as seen in a direction from the topsheet.

7. The absorbent article according to claim 6, wherein an inner end of the second part of the side portion of the outer cover layer is secured between the topsheet and the backsheet.

8. The absorbent article according to any one of the preceding claim 1, wherein the plurality of elastic elements in each leg cuff are constituted by at least 3 elastic elements.

9. The absorbent article according to claim 8, wherein the elastic elements are spaced apart in the transverse direction by from 1 mm to 8 mm.

10. The absorbent article according to any one of the preceding claim 1, wherein the outer leg cuff edge of the nonwoven barrier cuff strip is arranged at or inward of the fold line in the leg cuff fold as seen in the transverse direction.

11. The absorbent article according to claim 5, wherein the leg cuff portion of the nonwoven barrier cuff strip is attached to the second part of the side portion of the outer cover layer with an adhesive, the adhesive being uniformly distributed over an attachment area between the leg cuff portion of the nonwoven barrier cuff strip and the second part of the side portion of the outer cover layer.

12. The absorbent article according to claim 1, wherein the backsheet extends into the leg cuffs in an inner part of each leg cuff by at most 50% of the width of the leg cuff.

13. The absorbent article according to claim 12, wherein all or substantially all elastic elements of the plurality of elastic elements in each leg cuff are arranged in an outer part of the leg cuff which is free from the liquid impermeable backsheet.

14. The absorbent article according to claim 1, wherein the nonwoven barrier cuff strip is a single layer nonwoven material.

15. The absorbent article according to claim 1, wherein the elastic elements of the plurality of elastic elements are attached between the first and second parts of the leg cuff fold by means of adhesive.

16. The absorbent article according to claim 1, wherein one or more elastic elements being arranged in an inner part of the leg cuff fold has a greater tensile resistance than one or more elastic elements being arranged in an outer part of the leg cuff fold.

17. The absorbent article according to claim 16, wherein the one or more elastic elements in the inner part of the leg cuff fold has a linear density which is higher than a linear density of the one or more elastic elements being arranged in the outer portion of the leg cuff fold.

18. absorbent article having a first waist edge and a second waist edge (4)extending in a transverse direction of the absorbent article and a first side edge and a second side edge extending in a longitudinal direction of the absorbent article, the absorbent article comprising:
a liquid permeable topsheet having a topsheet first side edge and a topsheet second side edge;
a liquid impermeable backsheet having a backsheet first side edge and a backsheet second side edge;
an absorbent core disposed between the topsheet and the backsheet;
a nonwoven outer cover layer overlaying the backsheet and forming an outer surface of the absorbent article;
a pair of nonwoven barrier cuff strips extending in the longitudinal direction and being symmetrically arranged with one barrier cuff strip on each side of a longitudinal centre line, each nonwoven barrier cuff strip comprising a standing gather portion, being defined between a free standing gather edge extending in the longitudinal direction and a standing gather hinge line extending in the longitudinal direction, the barrier cuff strip being attached to the topsheet along the standing gather hinge line and having one or more pre-tensioned elastic elements arranged along the free standing gather edge;
a pair of standing gathers, the standing gathers comprising the standing gather portions of the barrier cuff strips;
a pair of leg cuffs extending in the longitudinal direction and being symmetrically arranged along the first and second side edges, each leg cuff being arranged outward of the standing gather portion of the corresponding barrier cuff strip; and
wherein
each leg cuff comprises:
a tube structure which extends in the transverse direction outward of the standing gather hinge line, having an outer tube edge defining the leg cuff fold edge and a leg cuff width in the transverse direction between the outer tube edge and the standing gather hinge line;
a plurality of spaced-apart pre-tensioned elastic elements extending in the longitudinal direction arranged inside the tube structure,
a leg cuff portion of the corresponding nonwoven barrier cuff strip, the leg cuff portion of the nonwoven barrier cuff strip being defined between the standing gather hinge line and an outer leg cuff edge of the nonwoven barrier cuff strip which outer leg cuff edge of the nonwoven barrier cuff strip is parallel to the outer tube edge of the tube structure, the leg cuff portion of the nonwoven barrier cuff strip overlying at least one elastic element of the plurality of elastic elements.

19. The absorbent article according to claim 18, wherein the tube structure is attached to the backsheet and/or the outer cover sheet.

20. The absorbent article according to claim 18, wherein the tube structure is arranged between the leg cuff portion of the corresponding nonwoven barrier cuff strip and the backsheet and/or the outer cover sheet.

21. The absorbent article according to 18, wherein the tube structure is arranged at least partly outside of the outer edge of the outer cover sheet in transverse direction.

22. The absorbent article according to claim 18, wherein the tube structure is formed by a continuous piece of sheet material being folded around the the plurality of elastic elements, and joined by a longitudinally extending joint to form the tube structure.
